(19) United States
(12) Patent Application Publication
(10) Pub. No.: US 2007/0160670 A1
(43) Pub. Date: Jul. 12, 2007

(54) Compression-coated tablets and manufacture thereof

(75) Inventors: Craig A. Judy, Mount Dandenong (AU); Peter H.R. Persicaner, Brighton (AU)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: ARROW NO. 7 LIMITED, London (GB)

(21) Appl. No.: 10/598,112

(22) PCT Filed: Feb. 15, 2005
(86) PCT No.: PCT/GB05/00541

§ 371 Date: Mar. 5, 2007

(30) Foreign Application Priority Data
Feb. 18, 2004 (GB) 0403628.1

(51) Int. Cl. A61K 31/405; A61K 9/24

(52) U.S. Cl. 424/472; 514/419

(57) ABSTRACT

Tablets for the treatment of migraine comprise a core, containing the active agent sumatriptan or a pharmaceutically-acceptable salt or ester thereof, and a mantle, free of sumatriptan. Tablets are prepared by compressing mantle components, free of the active agent, around a partially-compressed, active-containing core.

### TECHNICAL FIELD

The present invention relates to the field of tablet formulation. The invention is of application to active agents generally, but in particular, the invention relates to sumatriptan-containing tablets for the treatment of migraine, and methods of producing such tablets.

### BACKGROUND

Migraine is a common and debilitating condition that affects up to 10-15% of the population. The classical pattern of events in a migraine attack consists of an initial visual disturbance, followed, about 30 minutes later, by a severe throbbing headache, starting unilaterally, often with photophobia, nausea, vomiting and prostration.

Whilst the pathophysiology of migraine is not well understood, it is widely accepted that there is some relation between migraine and changes in cerebral blood flow. There is also strong evidence to implicate the neurotransmitter 5-hydroxytryptamine (5-HT), and many of the drugs that are effective in treating migraine are 5-HT receptor agonists or antagonists. One such drug is sumatriptan, an agonist of 5-HT_{1D} receptors, which receptors are predominantly expressed in cerebral blood vessels.

Many anti-migraine drugs have, until recently, been administered via injection, ie. as a liquid preparation. This has the advantage that the drugs are rapidly released into the systemic circulation and delivered to their sites of action—they can therefore act rapidly to relieve the symptoms of a migraine attack.

There are also, however, well-known disadvantages associated with the administration of drugs via injection; for example, the comparative difficulty in administration compared to a drug in tabletform, patient non-compliance, and safety issues relating to needles. Significant efforts have thus been made to develop oral tablets.

An oral tablet preparation of an anti-migraine drug must, as discussed above, rapidly release the drug into the stomach so that it can be absorbed into the systemic circulation and delivered to its site of action. An oral tablet preparation must also mask the unpleasant taste of the anti-migraine drug. Patients suffering a migraine attack are likely to experience nausea and vomiting—and are thus more averse than usual to the unpleasant taste of the drugs.

One solution to this taste-masking problem has been to coat oral tablet preparations with a layer of polymers, a process known as ‘film coating’. Film coating masks the taste of the drug when the tablet is placed into the mouth, but dissolves rapidly in the stomach so as to allow rapid release and absorption of the drug. Film-coated sumatriptan tablets are described in WO 92/15295, U.S. Pat. No. 6,368,627, U.S. Pat. No. 6,020,001 and U.S. Pat. No. 5,863,559.

Film coating is well established as a method of coating tablets and is used widely in the pharmaceutical industry. In brief, core tablets are compressed on a standard tablet press and transferred into a pan (typically a side vented coating pan). A mixture of, inter alia, polymers, colorants, opacifers, and plasticizers is mixed with water/solvents to create a coating dispersion. Warm air is passed through the bed of tablets while the coating dispersion is sprayed onto the tablets, forming the so-called “film”.

The process of film coating, however, requires expensive equipment for application of the layer of polymers, and also requires a drying step to fix the polymers onto the tablet. In addition, the process of film-coating (eg. by heating/moisture exposure) can stress the chemical composition/stability of the tablet and its active ingredient(s). Film coated tablets also require the use of a greater number of excipients, which can leave the active prone to reaction and degradation.

Alternative methods of masking the taste of unpleasant-tasting drugs in oral tablet preparations include the use of a mantle, free of drug, compressed around a core tablet containing the drug. Mantles of this type have been used to produce delayed release tablets (where the mantle is comprised of slowly dissolving material, delaying the release of the active), and sustained release tablets (the mantle acting as a semi-permanent barrier to the active contained in the core which must pass through the insoluble or partially soluble mantle). Other uses include a burst effect tablet, where an immediately-releasing mantle is coated over a sustained release core.

A disadvantage of these compression-coated tablets is the relatively large weight of the mantle, typically in excess of two times that of the core—hence, for a given core, adding a mantle will at least triple the size of the tablet.

It is desirable to develop a method of formulating these unpleasant tasting drugs, e.g. anti-migraine drugs, in an oral tablet preparation, said preparation having both taste-masking and rapid-release characteristics.

Accordingly, it is an object of the invention to provide an oral tablet preparation wherein the unpleasant taste of a drug is masked.

A further object is to provide an oral tablet preparation which allows rapid drug release.

It is a specific object of the invention to provide an oral tablet preparation of sumatriptan, wherein the unpleasant taste of sumatriptan is masked. A further specific object of the invention is to provide an oral tablet preparation of sumatriptan, wherein the unpleasant taste of sumatriptan is masked, and wherein release of sumatriptan from the tablet core is rapid.

### SUMMARY OF THE 5INVENTION

Accordingly, the present invention provides a tablet, comprising:-
(i) a core containing sumatriptan, and
(ii) a mantle, free of sumatriptan.

Tablets of the invention provide for rapid release of sumatriptan (reference to which indicates sumatriptan or a pharmaceutically acceptable salt or ester thereof) whilst the mantle of the tablet masks its unpleasant taste. Preferably, the weight ratio of mantle:core is equal to or less than 1.8:1, and more preferably the weight ratio of mantle:core is equal to or less than 1.5:1 and especially equal to or less than 1.3:1. The amount of sumatriptan is present in an amount effective to counter migraine, and suitably the core contains from 10-200 mg of sumatriptan.

More generally, the invention relates to use of compression-coating technology to mask an unpleasant taste in a tablet where the active needs to be rapidly released. Hence, in a further embodiment of the invention there is provided a tablet, comprising:-
(i) a core containing an active agent, and
(ii) a mantle, free of active agent.

Preferably, the weight ratio of mantle:core is equal to or less than 1.8:1, and more preferably the weight ratio of mantle:core is equal to or less than 1.5:1 and especially equal to or less than 1.3:1.

The invention additionally provides a method of producing a tablet, comprising the steps of:-
a) forming a core by:-
(i) placing a first amount of powder/granule in a press,
(ii) compressing said first amount of powder/granule to obtain a core, and
b) pressing a second amount of powder/granule around said core, thereby forming a mantle and obtaining the final tablet.

### BRIEF DESCRIPTION OF THE FIGURES

FIG. 1 shows the tablet press procedure used in the production of (i) a known compressed tablet and (ii) a compression-coated tablet of the invention.

FIG. 2 shows the dissolution profile of a tablet of the invention containing 25 mg of sumatriptan, as compared to a commercially-available Imitrex tablet containing 25 mg of sumatriptan.

FIG. 3 shows the dissolution profile of a tablet of the invention containing 50 mg of sumatriptan, as compared to a commercially-available Imitrex tablet containing 50 mg of sumatriptan.

FIG. 4 shows the dissolution profile of a tablet of the invention containing 100 mg of sumatriptan, as compared to a commercially-available Imitrex tablet containing 100 mg of sumatriptan.

### DETAILED DESCRIPTION OF THE INVENTION

Tablets of the invention are suitably formulated such that both the core and the mantle dissolve rapidly in the stomach. The dissolution can vary but a suitable dissolution profile is one in which at least 90% of the tablet is dissolved after 10, especially after 5, minutes, preferably 95% of the tablet is dissolved after 10, especially 5 minutes, as measured by the standard paddle method.

Generally, the core and the mantle disintegrate over substantially the same time period. The disintegration can be different however, and other tablets of the invention have a dissolution profile wherein the mantle is at least 95% dissolved and the core is at least 90% dissolved after 10, preferably after 5, minutes.

In preferred embodiments, the core of the tablet is usually comprised of one or more of the following components (referred to as the “core blend”):-
filler, binder, disintegrant, lubricant, active ingredient.

The filler can be any suitable filler that is pharmaceutically compatible with the active ingredient. Particular examples include, but are not limited to, Lactose, Calcium Phosphate, Calcium Sulfate, Compressible Sugar and derivatives thereof, Celluloses and derivatives thereof, Maltodextrin, and modified starches, or any combination thereof.

The binder can be any suitable binder that is pharmaceutically compatible with the active ingredient. Particular examples include, but are not limited to, Acacia, Carbomer, Carboxymethylcellulose Calcium (or Sodium), Cellulose Acetate Pthalate, MCC, Dextrates, Ethylcellulose, Gelatin, Liquid Glucose, Povidone, Starch (dry or as a paste), Guar Gum, Hydroxypropyl cellulose, HPMC, Maltodextrin. Poloaxmer and PEG, or any combination thereof.

The disintegrant can be any suitable disintegrant that is pharmaceutically compatible with the active ingredient. Particular examples include, but are not limited to, Alginic Acid, Calcium Phosphate Tribasic, Carboxymethylcellulose Calcium (or Sodium), MCC, Croscarmellose Sodium, Crospovidone, Docusate Sodium, Low substituted Hydroxypropyl Cellulose, magnesium aluminium Silicate, Polacrillan Potassium, Sodium Alginate, Sodium Bicarbonate, Sodium Starch Glycolate, Starch and Pregelatinized Starch, or any combination thereof.

The lubricant can be any suitable lubricant that is pharmaceutically compatible with the active ingredient. Particular examples include, but are not limited to, Calcium Stearate, Canola Oil, Hydrogenated Castor Oil, Colloidal Silicon Dioxide, Cottonseed Oil, Fumaric Acid, Glyceryl Monostearate, Glyceryl Palmitostearate, Magnesium Stearate, Mineral Oil, Poloxamer, Polyethylene Glycol, Polyoxyethylene Stearate, Polyvinyl Alcohol, Sodium Benzoate, Sodium Chloride, Sodium Lauryl Sulfate, Sodium Stearyl Fumarate, Stearic Acid, Talc, Hydrogenated Vegetable Oil, Glyceryl Dibehanate and Zinc Stearate, or any combination thereof.

The active ingredient is generally any active ingredient or mixture of active ingredients, though the invention is particularly suitable for actives which have an unpleasant taste and which are desired to be released rapidly. In specific embodiments, described in more details in examples below, the active is an anti-migraine drug, including 5-HT receptor agonists or antagonists or any combination thereof. Preferably, the active ingredient is sumatriptan. More preferably, the active ingredient is sumatriptan succinate. The tablets of the invention can each contain between 10 and 200 mg of active ingredient (free base), preferably between 25 and 100 mg, more preferably 25, 50 or 100 mg.

In preferred embodiments, the mantle is usually composed of one or more of the following components (referred to as the “mantle blend”):-
filler, binder, disintegrant, lubricant.

These components can be any of those mentioned above, or any combination thereof.

It is further optional that the core and the mantle are composed of substantially the same materials, apart from the active, which is in the core.

In addition, both the core and mantle blends may optionally comprise adsorbants and/or colorants. Any pharmaceutically acceptable colorant or adsorbant could be used.

The core and mantle blends are preferably prepared by dry blending (for ease of manufacture) but wet granulation or compaction granulation could be used.

In general, the components of the respective blends, except the lubricant, are blended together in a first blending step. More than one blending step may be required if colorant is to be added, so that the colorant is distributed uniformly within the blend. The lubricant is subsequently blended with the result of the first blending step.

In a particular embodiment, the core comprises, by weight:-
active ingredient: 1-40%
filler: 10-90%
binder: 2-60%
disintegrant: 1-60%
lubricant: 0.1-10%
adsorbants: 0-5%
colorants: 0-5%,
   and the mantle comprises, by weight:-
filler: 10-90%
binder: 2-60%
disintegrant: 1-60%
lubricant: 0.1-10%
adsorbants: 0-5%
colorants: 0-5%.

In another embodiment, the core comprises, by weight:-
sumatriptan 1-50%
filler: 10-90%
binder: 2-60%
disintegrant: 1-60%
lubricant: 0.1-10%
adsorbants: 0-5%
colorants: 0-5%
   and the mantle comprises, by weight:-
filler: 10-90%
binder: 2-60%
disintegrant: 1-60%
lubricant: 0.1-10%
adsorbants: 0-5%
colorants: 0-5%

In yet another embodiment, the core comprises by weight:-
sumatriptan 5-80%
filler: 10-90%
binder: 2-60%
disintegrant: 1-60%
lubricant: 0.1-10%
adsorbants: 0-5%
colorants: 0-5%
   and the mantle comprises, by weight:-
filler: 10-90%
binder: 2-60%
disintegrant: 1-60%
lubricant: 0.1-10%
adsorbants: 0-5%
colorants: 0-5%

In a preferred embodiment, the core tablet comprises:-
Sumatriptan Succinate
Microcrystalline Cellulose
Croscarmellose Sodium
Purified Water
Magnesium Stearate
   and the mantle of the tablet comprises:-
Lactose
Microcrystalline Cellulose
Croscarmellose Sodium
Iron Oxide Red
Magnesium Stearate.

An advantage of specific tablets of the invention is that they act rapidly to relieve the symptoms of a migraine attack, as the active agent in the tablets is rapidly released into the stomach, so that it can be absorbed into the systemic circulation and delivered to its site of action. The tablets of the invention have additionally been shown to have improved dissolution profiles compared to commercially-available oral tablet preparations of sumatriptan, as evidenced in Example 4.

Tablets of the invention can be prepared by:-
(A) forming a core by:
(i) placing a first amount of powder/granule in a press,
(ii) compressing said first amount of powder/granule to obtain a core, and
(B) forming a mantle around the core by:-
(i) placing a second amount of powder/granule in a press,
(ii) placing said core onto said second amount of powder/granule,
(iii) placing a third amount of powder/granule on top of the core and the second amount of powder/granule, and
(iv) compressing (iii) so as to obtain the final tablet.

The core compression in Step A is suitably carried out at a pressure of from 0.5-5 tons, resulting in a partially-compressed core. This core is sufficiently compressed to remain intact during subsequent processing steps, and prior to further compression in Step B.

The compression in Step B is generally carried out at a higher pressure, suitably carried out at a pressure of from 0.5-10 tons, to yield the final tablet.

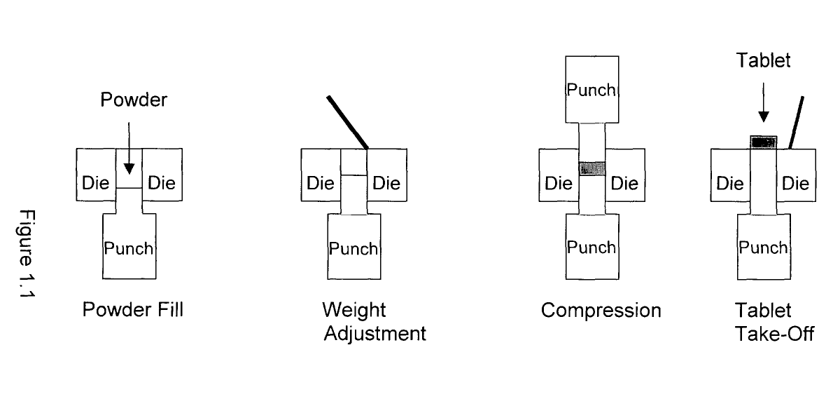

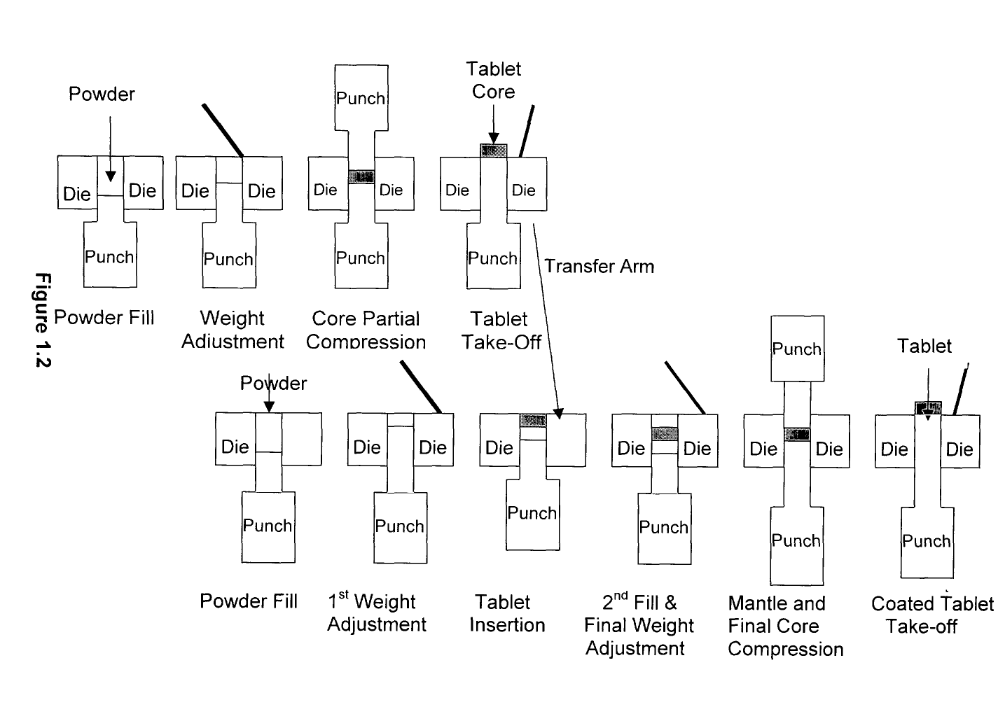

In a method for making tablets of the invention, the tablets are formed using the process shown schematically in FIG. 1. Specifically, the tablet is formed in two stages. First, a core is formed from a core blend containing active. Subsequently, a mantle blend, free of active, is compressed around this core to form the final tablet.

To form the core, a given amount of a first powder/granule (ie. the core blend, containing active) is compressed on a conventional tablet press. Preferably, the compression is carried out at a pressure of 0.5-5 tons, to form a partially-compressed core. This partial compression hold the components of the core together, but is not compressed to its final hardness/thickness.

To form the final tablet, a given amount of a second powder/granule (ie. the mantle blend, free of active) is placed in a press, onto which the core is placed. The core is then covered with an additional amount of a third powder (also mantle blend), and all components forming the tablet are then compressed to the final hardness and thickness. Preferably, the compression is carried out at a pressure of 0.5-10 tons.

The tablets of the invention may be formed by a process wherein both the core and mantle blends are placed onto the two sides of a single machine (e.g. a Manesty Dry-Cota tablet press). The core blend is again partially compressed (compressed, but not to its final hardness/thickness, only sufficient to hold together until it is transferred) on the core side of the press and then transferred to the mantle side of the press where it is sandwiched in the mantle blend. This core/mantle blend is then compressed into the final, compression-coated tablet.

Preferably, the tablets of the invention have a friability index of less than 1%, more preferably less than 0.8%.

We have found that, following the invention, it is possible for the core of the tablet to be produced using approximately half the quantity of the excipients used in other commercially-available sumatriptan tablets. Surprisingly, and despite the small quantity of excipients used, adequate disintegration/dissolution of the core is achieved.

Furthermore, we have also found that good mantle coverage can be achieved with a low ratio (by weight) of mantle:core. For example, whereas a typical prior art weight ratio of mantle:core would be 2:1, a good mantle coverage is achieved in the invention with a ratio as low as approximately 1.15:1.

The invention is now described and illustrated in the following examples.

### EXAMPLE 1

The core tablet (Items 1-8) and mantle (Items 9-13) were formulated as shown in Table 1 below.

TABLE 1
Formulation of core and mantle blends.

| | | mg/tab | | |
|---|---|---|---|---|
| Item | Ingredient | 25 mg | 50 mg | 100 mg |
| 1 | Sumatriptan Succinate | 35.00 | 70.00 | 140.00 |
| 2 | Microcrystalline Cellulose | 18.20 | 36.40 | 72.80 |
| 3 | Croscarmellose Sodium | 2.80 | 5.60 | 11.20 |
| 4 | Purified Water | q.s. | q.s. | q.s. |
| 5 | Purified Water | q.s. | q.s. | q.s. |

Add Drys

| | | | | |
|---|---|---|---|---|
| 6 | Microcrystalline Cellulose | 10.5 | 21 | 42 |
| 7 | Croscarmellose Sodium | 2.8 | 5.6 | 11.2 |
| 8 | Magnesium Stearate | 0.7 | 1.4 | 2.8 |
| | Total Tablet Core Weight | 70 | 140 | 280 |

Mantle Blend

| | | | | |
|---|---|---|---|---|
| 9 | Lactose | 41.75 | 83.5 | 167 |
| 10 | Microcrystalline Cellulose | 35.75 | 71.5 | 143 |
| 11 | Croscarmellose Sodium | 1.6 | 3.2 | 6.4 |
| 12 | Colorant ** | 0.1 | 0.2 | 0.4 |
| 13 | Magnesium Stearate | 0.8 | 1.6 | 3.2 |
| | Total Tablet Weight | 150 | 300 | 600 |

** Optional colorant. If not used, replace with lactose.

In these particular formulations, Lactose acts as a filler, Microcrystalline Cellulose acts as a binder/disintegrant and filler, Croscarmellose Sodium acts as a disintegrant, and the Magnesium Stearate acts as a lubricant.

### EXAMPLE 2

With reference to Table 1 of Example 1, tablets were manufactured as follows:-

Items 1-3 were transferred into a high shear mixer/granulator after being pre-screened. The powders were mixed for 2 minutes at settings of High Mix and High Chop. Items 4-5 were then used to granulate the material under the same settings. After a suitable granule was achieved, this was discharged into the bowl of a fluid bed dryer and the material dried at a temperature of 50° C. to a Loss On Drying (LOD) reading of less than 2%.

The dried granule was milled through a screen to remove the oversized particles (a screen size of approximately 425-1400 microns can be used, preferably 600 microns).

The milled granule was transferred to an appropriately sized tumble blender, along with Items 6 & 7 (after pre-screening). These were blended for 10 minutes prior to addition of Item 8 (again pre-screened). Final blending was effected for 5 minutes. This completed the so-called “core blend”.

Separately, Items 9-12 (pre-screened) were transferred into an appropriately sized tumble blender and blended for 10 minutes. If Item 12 (the optional colorant) is used, an intermediate blending step with one of the other ingredients, may be used to properly disperse the colorant into the blend. Pre-screened Item 13 was added to the blender and final mixing effected for 5 minutes. This completed the so-called “mantle blend.”

The core and mantle blends were then placed onto the two sides of a Manesty Dry-Cota tablet press. The core blend was partially compressed (not to its final hardness/thickness, but sufficient to hold together until transferred) on the core side of the press and then transferred to the mantle side of the press where it was sandwiched in a bed of the mantle blend. This core/mantle blend was then compressed into the final, compression-coated tablet. Other methods of compression coating can be used, e.g. by compressing core tablets on one machine and then transferring them to a separate machine for compression coating.

### EXAMPLE 3

Tablets A, B and C, containing 25 mg, 50 mg, and 100 mg of sumatriptan respectively, were made in accordance with Example 1.

The dissolution profiles of tablets A-C were tested using the standard Paddle Method (see European Pharmacopoeia) and the results are shown in Tables 2-4. These tables also provide comparative dissolution profiles of commercially-available sumatriptan tablets manufactured by GlaxoSmithKline (Imitrex).

TABLE 2
Comparison of Dissolution Profiles by UV in 0.1 N HCl
Tablet A (25 mg) vs Imitrex Tablets 25 mg (GSK)

| | | Time Points (Minutes) | | | | |
|---|---|---|---|---|---|---|
| | Tablet | 0 | 5 | 10 | 15 | 20 |
| Tablet A | 1 | 0 | 97.9 | 98.1 | 98.0 | 97.9 |
| 25 mg | 2 | 0.0 | 100.5 | 100.8 | 100.7 | 100.6 |
| | 3 | 0.0 | 99.1 | 99.7 | 99.6 | 99.4 |
| | 4 | 0.0 | 98.0 | 98.9 | 98.8 | 98.8 |
| | 5 | 0.0 | 97.8 | 99.4 | 98.8 | 98.7 |
| | 6 | 0.0 | 99.6 | 99.8 | 99.8 | 99.7 |
| | 7 | 0.0 | 100.4 | 100.9 | 100.8 | 100.8 |
| | 8 | 0.0 | 98.3 | 99.2 | 99.1 | 99.1 |
| | 9 | 0.0 | 99.8 | 100.4 | 100.4 | 100.5 |
| | 10 | 0.0 | 99.3 | 100.1 | 100.1 | 100.3 |
| | 11 | 0.0 | 97.6 | 98.5 | 98.5 | 98.6 |
| | 12 | 0.0 | 98.1 | 99.0 | 99.1 | 99.2 |
| | Mean | 0 | 99 | 100 | 100 | 100 |
| Imitrex | 1 | 0.0 | 53.5 | 97.8 | 101.5 | 101.5 |
| 25 mg | 2 | 0.0 | 67.1 | 101.2 | 101.9 | 102.0 |
| | 3 | 0.0 | 67.4 | 101.2 | 101.7 | 101.7 |
| | 4 | 0.0 | 58.1 | 98.1 | 102.1 | 102.2 |
| | 5 | 0.0 | 71.1 | 100.1 | 100.6 | 100.6 |
| | 6 | 0.0 | 76.2 | 99.9 | 99.7 | 99.6 |
| | 7 | 0.0 | 61.3 | 98.6 | 100.1 | 100.1 |
| | 8 | 0.0 | 65.6 | 98.8 | 99.4 | 99.6 |
| | 9 | 0.0 | 61.2 | 97.3 | 97.9 | 97.9 |
| | 10 | 0.0 | 60.6 | 100.9 | 101.6 | 101.6 |
| | 11 | 0.0 | 78.8 | 99.3 | 99.6 | 99.6 |
| | 12 | 0.0 | 63.2 | 100.7 | 101.3 | 101.4 |
| | Mean | 0 | 65 | 100 | 101 | 101 |

TABLE 3
Comparison of Dissolution Profiles bv UV in 0.1 N HCl
Tablet B (50 mg) vs Imitrex Tablets 50 mg (GSK)

| | | Time Points (Minutes) | | | | |
|---|---|---|---|---|---|---|
| | Tablet | 0 | 5 | 10 | 15 | 20 |
| Tablet B | 1 | 0.0 | 99.8 | 99.8 | 99.6 | 99.3 |
| 50 mg | 2 | 0.0 | 101.1 | 100.7 | 100.2 | 100.0 |
| | 3 | 0.0 | 99.2 | 100.0 | 99.7 | 99.6 |
| | 4 | 0.0 | 99.9 | 100.7 | 100.6 | 100.5 |
| | 5 | 0.0 | 91.7 | 99.6 | 99.6 | 99.5 |
| | 6 | 0.0 | 99.0 | 99.9 | 99.7 | 99.7 |
| | 7 | 0.0 | 99.4 | 99.5 | 99.5 | 99.1 |
| | 8 | 0.0 | 100.5 | 100.2 | 100.1 | 99.9 |
| | 9 | 0.0 | 99.0 | 99.7 | 99.6 | 99.4 |
| | 10 | 0.0 | 99.6 | 100.6 | 100.5 | 100.3 |
| | 11 | 0.0 | 91.4 | 99.5 | 99.6 | 99.3 |
| | 12 | 0.0 | 98.5 | 99.8 | 99.5 | 99.6 |
| | Mean | 0 | 98 | 100 | 100 | 100 |
| Imitrex | 1 | 0.0 | 90.2 | 99.5 | 99.7 | 99.8 |
| 50 mg | 2 | 0.0 | 85.9 | 99.2 | 99.4 | 99.5 |
| | 3 | 0.0 | 86.9 | 99.7 | 99.9 | 100.0 |
| | 4 | 0.0 | 93.7 | 97.7 | 97.7 | 97.7 |
| | 5 | 0.0 | 74.6 | 98.0 | 98.4 | 98.4 |
| | 6 | 0.0 | 94.0 | 98.2 | 98.3 | 98.3 |
| | 7 | 0.0 | 70.1 | 102.4 | 103.0 | 102.9 |
| | 8 | 0.0 | 96.5 | 99.5 | 99.3 | 99.3 |
| | 9 | 0.0 | 86.8 | 99.6 | 99.7 | 99.7 |
| | 10 | 0.0 | 77.9 | 102.2 | 102.6 | 102.6 |
| | 11 | 0.0 | 93.3 | 100.6 | 100.6 | 100.6 |
| | 12 | 0.0 | 88.0 | 101.1 | 101.3 | 101.2 |
| | Mean | 0 | 87 | 100 | 100 | 100 |

TABLE 4
Comparison of Dissolution Profiles by UV in 0.1 N HCl
Tablet C (100 mg) vs Imitrex Tablets 100 mg (GSK)

| | | Time Points (Minutes) | | | | |
|---|---|---|---|---|---|---|
| | Tablet | 0 | 5 | 10 | 15 | 20 |
| Tablet C | 1 | 0.0 | 96.8 | 98.3 | 98.3 | 98.3 |
| 100 mg | 2 | 0.0 | 97.3 | 98.4 | 98.1 | 99.1 |
| | 3 | 0.0 | 97.9 | 99.4 | 99.0 | 99.6 |
| | 4 | 0.0 | 97.2 | 98.5 | 98.3 | 98.7 |
| | 5 | 0.0 | 97.0 | 98.2 | 98.0 | 98.1 |
| | 6 | 0.0 | 97.4 | 98.1 | 97.8 | 97.8 |
| | 7 | 0.0 | 98.9 | 98.8 | 99.6 | 99.6 |
| | 8 | 0.0 | 100.1 | 101.1 | 100.6 | 100.7 |
| | 9 | 0.0 | 100.4 | 101.0 | 100.7 | 100.8 |
| | 10 | 0.0 | 100.4 | 101.6 | 101.8 | 101.4 |
| | 11 | 0.0 | 99.8 | 101.3 | 101.5 | 101.1 |
| | 12 | 0.0 | 99.6 | 100.9 | 101.3 | 101.4 |
| | Mean | 0 | 99 | 100 | 100 | 100 |
| Imitrex | 1 | 0.0 | 94.0 | 100.0 | 100.1 | 99.9 |
| 100 mg | 2 | 0.0 | 98.1 | 101.2 | 101.1 | 101.0 |
| | 3 | 0.0 | 97.6 | 99.5 | 99.6 | 99.8 |
| | 4 | 0.0 | 95.0 | 94.8 | 96.7 | 96.7 |
| | 5 | 0.0 | 95.4 | 96.8 | 96.8 | 96.7 |
| | 6 | 0.0 | 95.0 | 96.8 | 97.1 | 96.9 |
| | 7 | 0.0 | 93.1 | 98.3 | 99.1 | 98.6 |
| | 8 | 0.0 | 97.4 | 101.0 | 101.3 | 101.0 |
| | 9 | 0.0 | 99.9 | 101.8 | 101.8 | 101.4 |
| | 10 | 0.0 | 98.0 | 99.2 | 99.3 | 99.1 |
| | 11 | 0.0 | 95.9 | 98.0 | 98.2 | 98.2 |
| | 12 | 0.0 | 95.7 | 98.5 | 98.2 | 98.3 |
| | Mean | 0 | 96 | 99 | 99 | 99 |

As can be seen from Table 2, tablets of the invention containing 25 mg of sumatriptan show rapid dissolution, with 99% (mean average) dissolution after 5 minutes and 100% dissolution after 10 minutes. This contrasts favourably with the Imitrex 25 mg tablet, which, under the same conditions, shows only 65% mean average dissolution after 5 minutes, and 100% dissolution after 10 minutes.

Under the same conditions, tablets of the invention containing, respectively, 50 mg and 100 mg of sumatriptan show similarly impressive dissolution profiles (see Tables 3 and 4).

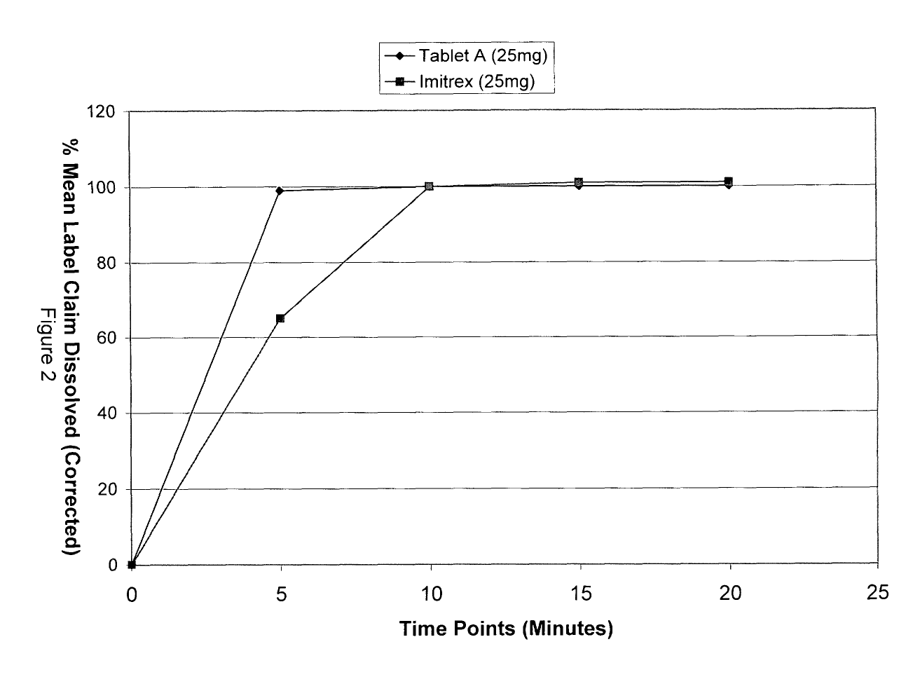

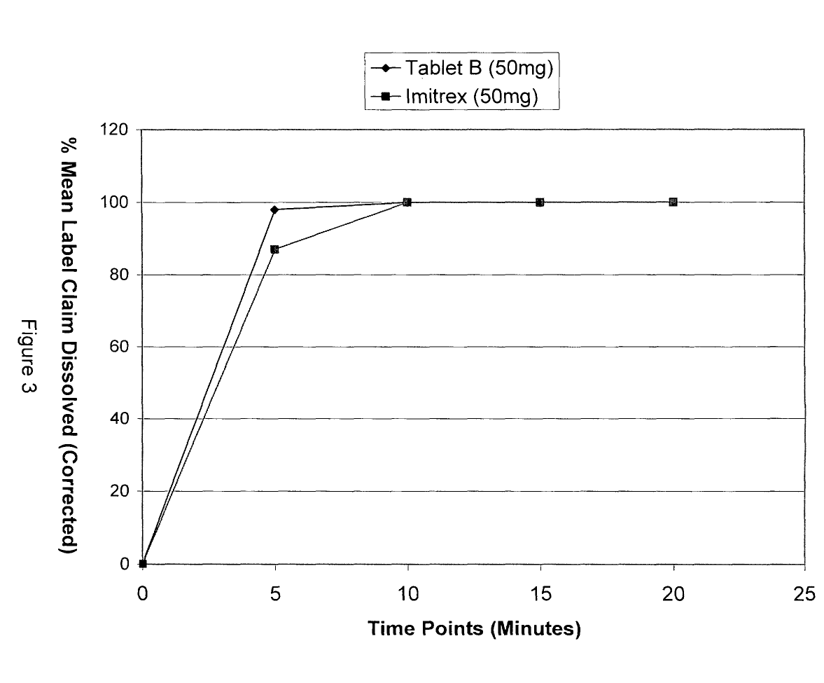

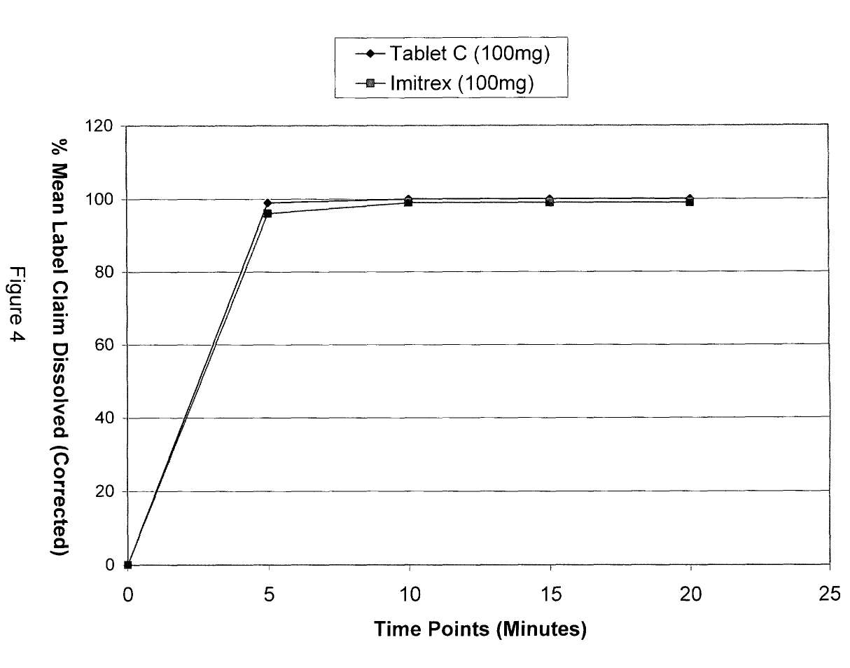

The data in Tables 2-4 is represented graphically in FIGS. 2-4.

### EXAMPLE 4

A bio-study was carried out and showed the 100 mg tablets of Example 3 were bio-equivalent to the branded Glaxo product (Imitrex®). This study also showed that there was no report by patients of a unpleasant-taste when the tablets were taken. The study thus confirmed rapid release of active and adequate taste-masking by tablets of the invention.

1. A tablet, comprising:
(i) a core containing sumatriptan, and
(ii) a rapid release mantle, free of sumatriptan, wherein the mantle entirely surrounds the core.

2. A tablet according to claim 1, wherein the weight ratio of mantle:core is equal to or less than 1.8:1.

3. A tablet according to claim 1, wherein the weight ratio of mantle:core is equal to or less than 1.5:1.

4. A tablet according to claim 1, wherein the core contains from 10-200 mg of sumatriptan.

5. A tablet according to claim 1, wherein:
(i) the core comprises sumatriptan, a filler, a binder, a disintegrant and a lubricant, and
(ii) the mantle comprises a filler, a binder, a disintegrant and a lubricant.

6. A tablet according to claim 5, wherein the core and the mantle further comprise adsorbants and/or colorants.

7. A tablet according to claim 6, wherein the core comprises, by weight:
sumatriptan: 1-40%
filler: 10-90%
binder: 2-60%
disintegrant: 1-60%
lubricant: 0.1-10%
adsorbants: 0-5%
colorants: 0-5%
and the mantle comprises, by weight:
filler: 10-90%
binder: 2-60%
disintegrant: 1-60%
lubricant: 0.1-10%
adsorbants: 0-5%
colorants: 0-5%

8. A tablet according to claim 6, wherein the core comprises by weight:
sumatriptan: 1-50%
filler: 10-90%
binder: 2-60%
disintegrant: 1-60%
lubricant: 0.1-10%
adsorbants: 0-5%
colorants: 0-5%
and the mantle comprises, by weight:
filler: 10-90%
binder: 2-60%
disintegrant: 1-60%
lubricant: 0.1-10%
adsorbants: 0-5%
colorants: 0-5%

9. A tablet according to claim 6, wherein the core comprises by weight:
sumatriptan: 5-80%
filler: 10-90%
binder: 2-60%
disintegrant: 1-60%
lubricant: 0.1-10%
adsorbants: 0-5%
colorants: 0-5%
and the mantle comprises, by weight:
filler: 10-90%
binder: 2-60%
disintegrant: 1-60%
lubricant: 0.1-10%
adsorbants: 0-5%
colorants: 0-5%

10. A tablet according to claim 1, wherein, apart from the sumatriptan in the core, the core and the mantle comprises substantially the same materials.

11. A tablet according to claim 1, wherein both the core and the mantle dissolve rapidly in the stomach.

12. A tablet according to claim 11, wherein at least 90% of the tablet is dissolved after 10 minutes.

13. A tablet according to claim 1, wherein the core and the mantle disintegrate over substantially the same time period.

14. A tablet according to claim 13, wherein the mantle is at least 95% dissolved and the core is at least 90% dissolved after 10 minutes.

15. A method of producing a tablet according to claim 1, comprising the steps of:
(a) forming a core by:
(i) placing a first amount of powder/granule in a press,
(ii) compressing said first amount of powder/granule to obtain a core, and
(b) pressing a second amount of powder/granule around said core, thereby forming a mantle and obtaining the final tablet.

16. A method of producing a tablet according to claim 15, comprising the steps of:
(a) forming a core by:
(i) placing a first amount of powder/granule in a press,
(ii) compressing said first amount of powder/granule to obtain a core, and
(b) forming a mantle around the core by:
(i) placing a second amount of powder/granule in a press,
(ii) placing said core onto said second amount of powder/granule,
(iii) placing a third amount of powder/granule on top of the core and the second amount of powder/granule, and
(iv) compressing (iii) so as to obtain the final tablet.

17. A method according to claim 15, wherein the compression in Step (a) is carried out at pressure of from 0.5-5 tons.

18. A method according to claim 15, wherein the compression in Step (b) is carried out at a pressure from 0.5-10 tons.

19. A method according to claim 15, wherein the first amount of powder/granule comprises sumatriptan, a filler, a binder, a disintegrant and a lubricant.

20. A method according to claim 19, wherein the first amount of powder/granule further comprises an adsorbant and/or a colorant.

21. A method according to claim 15, wherein the first amount of powder/granule comprises, by weight:
sumatriptan: 1-40%
filler: 10-90%
binder: 2-60%
disintegrant: 1-60%
lubricant: 0.1-10%
adsorbants: 0-5%
colorants: 0-5%

22. A method according to claim 15, wherein the first amount of powder/granule comprises, by weight:
sumatriptan: 1-50%
filler: 10-90%
binder: 2-60%
disintegrant: 1-60%
lubricant: 0.1-10%
adsorbants: 0-5%
colorants: 0-5%

23. A method according to claim 15, wherein the first amount of powder/granule comprises, by weight:
sumatriptan: 5-80%
filler: 10-90%
binder: 2-60%
disintegrant: 1-60%
lubricant: 0.1-10%
adsorbants: 0-5%
colorants: 0-5%

24. A method according to claim 15, wherein the second and/or third amounts of powder/granule comprise a filler, a binder, a disintegrant and a lubricant.

25. A method according to claim 24, wherein the second and/or third amounts of powder/granule further comprise an adsorbant and/or a colorant.

26. A method according to claim 15, wherein the second and/or third amounts of powder/granule comprise, by weight:
filler: 10-90%
binder: 2-60%
disintegrant: 1-60%
lubricant: 0.1-10%
adsorbants: 0-5%
colorants: 0-5%

27. A method according to claim 15, wherein Step (a) results in a partially-compressed core, which core is then further compressed in Step (b).
